(19) United States
(12) Patent Application Publication
(10) Pub. No.: US 2017/0007637 A1
(43) Pub. Date: Jan. 12, 2017

(54) PHARMACOLOGICAL OPHTHALMIC COMPOSITION FOR USE IN THE CORRECTION OF PRESBYOPIA AND ITS ADMINISTRATION

(71) Applicant: ORASIS PHARMACEUTICALS LTD., Herzliya (IL)

(72) Inventors: Claes G. FEINBAUM, Herzliya (IL); Sudhir PATEL, Stirling (GB); Franc SALAMUN, Kostanjevica na Krasu (SI)

(21) Appl. No.: 15/117,798

(22) PCT Filed: Feb. 11, 2014
(86) PCT No.: PCT/SI2014/000008

§ 371 Date: Aug. 10, 2016

(51) Int. Cl. A61K 31/728; A61K 9/00; A61K 9/08; A61K 31/4178; A61K 31/196

(52) U.S. Cl. CPC A61K 31/728; A61K 31/4178; A61K 31/196; A61K 9/08; A61K 9/0048; A61K 9/0051

(57) ABSTRACT

The object of the invention is to provide a pharmacological ophthalmic composition for use in the correction of presbyopia as a drop introduced onto the surface of the eye and surrounding soft tissue (drops) or as an implant surgically introduced into the subconjunctival space with sustained slow release application.

The object of the invention is to provide a pharmacological ophthalmic composition for use in the correction of presbyopia as a drop introduced onto the surface of the eye and surrounding soft tissue (drops) or as an implant surgically introduced into the eye with sustained slow release application. With drops, or implant, a reduction of pupil size is achieved, leading to an increase in depth of field, a fall in the magnitude of the ocular high order optical aberrations and improved near and distance unaided visual acuity. The invention belongs to class A 61K45/06, A61K 31/4178 and A61P 27/10 of international patent classification.

When a young emmetrope fixates on a near object two main changes occur in the eye: accommodation and miosis. Accommodation is a change in crystalline lens refractive power. Lens becomes rounder, by which its refractive power increases. Miosis is a decrease in pupil size by which depth of focus increases and high order aberrations decrease.

Both, miosis and accommodation occur under the influence of the parasympathetic nervous system. Parasympathomimetic, acetylcholine, binding to muscarinic receptors, induces muscular contraction of the ciliary muscle and the sphincter of the pupil.

Presbyopia is decreasing age-related ability to focus on objects at close distances because of a gradual loss of accommodative amplitude. Soon after the age of fifty, lens loses all its ability to undergo accommodative optical changes. Measured subjectively, there is a progressive loss of accommodative amplitude until about the age of fifty, where it remains at about 1 diopter. This is depth of focus due to aberrations and miosis, defined as pseudo accommodation.

Presbyopia has been corrected with the use of spectacles, contact lenses or intra-ocular implants, and corneal ablation or inlays. The surgical methods that have been proposed to correct presbyopia do not completely restore the natural accommodative functionality of the eye that has been reduced either by natural ageing or by other means. Pharmacological treatments have been proposed to restore the natural loss of the accommodative functionality of the eye that leads to presbyopia. According to EP 1 938 839, a combination of parasympathomimetics and non-steroidal anti-inflammatory agents where the parasympathomimetic in use is pilocarpine (or its salts) in concentration from 1% to 2%. These concentrations of the parasympathomimetic can lead to undesirable side effects.

It is also known that delivery of an ophthalmic composition with sustained-release by way of an intravitreal microinsert near the base of the eye is efficient and advantageous. Such treatment is described in the WO 2011/079123 A1 document (description of the procedure set out in the attached pat. document (WO2011/079123 A1). The advantage of the microinsert is that the slowly released ingredients remain in the eye, are not lost via the natural drainage channels associated with fluids introduced onto the ocular surface. The microinsert saves the user (patient) time and effort by avoiding repeat instillation of drops every so often. The present invention relates to the ophthalmic composition for use in the correction of presbyopia without harmful influences as stated in the other documents.

Ophthalmic composition according to the invention contains the following: Sodium Hyaluronate from 0.1% to 0.9%, Diclofenac Sodium from 0.006% to 0.012% and Pilocarpine Hydrochloride from 0.2% to 0.4%. The composition of the invention extends to encompass other constituents that may be added, modified, substituted or removed to improve comfort and overall efficacy.

The modus operandi of the invention is as follows.

The size of the pupil reduces as a consequence of the action of the constituents of the drop on the muscle fibres of the natural iris. Inducing miosis, depth of focus increases and the magnitude of high order aberrations decrease, improving uncorrected near and distance visual acuity.

The advantage of the composition is i) the low dosage ii) anti-inflammatory action iii) comfort. A literature search does not reveal any known long term harmful effects of any of the constituents in the noted concentrations on the eye. This is supported by our observations based on over 100 cases (62 subjects).

The drops are proposed for use on persons of generally good heath with small distance optical prescriptions, those that previously had laser eye surgery of the cornea (LASIK or PRK), those that previously had routine cataract surgery and implanted with standard monofocal intra-ocular implant lens. One or two drops are introduced onto the ocular surface, the improvement in distance and near vision is normally reported after a few minutes and the effect can last up to 8 hours.

The slow release implant is surgically introduced into subconjunctival space by an ophthalmic surgeon after s/he has decided that the patient would benefit from the implant following provocative testing using the topical drops. The constituents passively reach the iris, interact with the muscle fibres of the iris changing the size of the pupil. This action leads to an increase in depth of field and improvement in the distance and near vision as noted herein.

1-3. (canceled)

4. A pharmaceutical composition comprising sodium hyaluronate in a concentration ranging from 0.1% to 0.9%, diclofenac sodium in a concentration ranging from 0.006% to 0.012%, and pilocarpine hydrochloride in a concentration ranging from 0.2% to 0.4%.

5. A method of treating presbyopia comprising administering to a person suffering therefrom a therapeutically effective amount of the pharmaceutical composition of claim 4.

6. The method of claim 5, wherein the pharmaceutical composition is administered as topical drops.

7. The method of claim 6, wherein the pharmaceutical composition is administered as one to two topical drops introduced onto the ocular surface of the eye.

8. The method of claim 8, wherein the pharmaceutical composition is administered every eight hours.

9. The method of claim 7, wherein the pharmaceutical composition is administered every eight hours.

10. An intravitreal microinsert comprising the pharmaceutical composition of claim 4.

11. The intravitreal microinsert of claim 10, wherein the microinsert is configured to deliver the pharmaceutical composition over a sustained period of time.

12. A method of treating presbyopia comprising surgically introducing an intravitreal microinsert according to claim 10 to the subconjunctival space of an eye of a person suffering therefrom.

13. The method of claim 10, wherein the person suffering from presbyopia has previously been provocatively tested with topical drops comprising the pharmaceutical composition.

14. A method of treating presbyopia comprising surgically introducing an intravitreal microinsert according to claim 11 to the subconjunctival space of an eye of a person suffering therefrom.

15. The method of claim 14, wherein the person suffering from presbyopia has previously been provocatively tested with topical drops comprising the pharmaceutical composition.

16. The method of claim 5, wherein the person suffering from presbyopia has previously had at least one of laser eye surgery of the cornea, cataract surgery, or implantation of a standard monofocal intraocular implant lens.

17. The method of claim 12, wherein the person suffering from presbyopia has previously had at least one of laser eye surgery of the cornea, cataract surgery, or implantation of a standard monofocal intraocular implant lens.

18. The method of claim 14, wherein the person suffering from presbyopia has previously had at least one of laser eye surgery of the cornea, cataract surgery, or implantation of a standard monofocal intraocular implant lens.
